(19) United States
(12) Patent Application Publication
(10) Pub. No.: US 2025/0302301 A1
(43) Pub. Date: Oct. 2, 2025

(54) FACILITATING OPHTHALMIC SURGERY USING AUTOMATED DETECTION OF PURKINJE IMAGES

(71) Applicant: Alcon Inc., Fribourg (CH)

(72) Inventors: Lu YIN, Keller, TX (US); Vignesh SURESH, Woodland Hills, CA (US); Ramesh SARANGAPANI, Coppell, TX (US)

(21) Appl. No.: 19/064,050

(22) Filed: Feb. 26, 2025

Related U.S. Application Data
(60) Provisional application No. 63/570,404, filed on Mar. 27, 2024.

(51) Int. Cl. A61B 3/13

(52) U.S. Cl. CPC A61B 3/13

(57) ABSTRACT

A system includes an ophthalmic microscope including first and second illuminator optics configured to emit light onto an eye of a patient. The system further includes a controller coupled to the first and second illuminator optics. The controller is configured to operate in a first mode in which light emitted by the first illuminator optics and light emitted by the second illuminator optics has a first configuration. The controller is configured to operate in a second mode in which the light emitted by the first illuminator optics and the light emitted by the second illuminator optics are configured to enhance visibility of one or more Purkinje images projected onto the eye of the patient by the first illuminator optics relative to the first configuration. Registration of the optical axis of the eye, robotic alignment, and autofocusing may also be performed using Purkinje images.

### CROSS-REFERENCE TO RELATED APPLICATIONS

This application claims priority to U.S. Provisional Application No. 63/570,404, filed on Mar. 27, 2024, which is hereby incorporated by reference in its entirety.

### INTRODUCTION

The present disclosure relates generally to providing imaging during ophthalmic surgery, such as cataract surgery.

The human eye receives light through a clear outer portion called the cornea and focuses the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished light that is transmitted to the retina. This deficiency in the lens of the eye is medically known as a cataract. In addition, the crystalline lens may lose accommodation skills with age, which is called presbyopia. An accepted treatment for those conditions is the surgical removal of the crystalline lens followed by a replacement by an artificial intraocular lens (IOL).

### SUMMARY

In certain embodiments, a system includes an ophthalmic microscope including first illuminator optics and second illuminator optics configured to emit light onto an eye of a patient. The system further includes a controller coupled to the first illuminator optics and the second illuminator optics. The controller is configured to operate in a first mode in which light emitted by the first illuminator optics and light emitted by the second illuminator optics has a first configuration. The controller is configured to operate in a second mode in which the light emitted by the first illuminator optics and the light emitted by the second illuminator optics are configured to enhance visibility of one or more Purkinje images projected onto the eye of the patient by the first illuminator optics relative to the first configuration.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example ophthalmic system with which ophthalmic treatments are performed in an operating environment, in accordance with certain embodiments.

FIG. 2A illustrates components of an ophthalmic microscope and Purkinje images, in accordance with certain embodiments.

FIG. 2B illustrates an arrangement of lights of an ophthalmic microscope.

FIG. 2C is a schematic representation of an image of an eye with aligned Purkinje images.

FIG. 3A illustrates a relative orientation of an eye resulting in misaligned Purkinje points.

FIG. 3B is a schematic representation of an image of an eye with misaligned Purkinje images.

FIG. 4 is a schematic representation of an ophthalmic microscope configured to perform light modulation to facilitate visualization of Purkinje images in accordance with certain embodiments.

FIG. 5 is a process flow diagram of a method for performing light modulation to facilitate visualization of Purkinje images in accordance with certain embodiments.

FIG. 6 is a process flow diagram of a method for performing coded modulation to facilitate visualization of Purkinje images in accordance with certain embodiments.

FIG. 7 is a schematic representation of an ophthalmic microscope configured to perform automated registration with respect to Purkinje images in accordance with certain embodiments.

FIG. 8 is a process flow diagram of a method for performing automated registration with respect to Purkinje images in accordance with certain embodiments.

FIG. 9 is a schematic representation of an ophthalmic microscope configured to activate a robotic actuator according to identified Purkinje images in accordance with certain embodiments.

FIG. 10 is a process flow diagram of a method for activating a robotic actuator according to identified Purkinje images in accordance with certain embodiments.

FIG. 11 is a process flow diagram of a method for performing automated focusing according to identified Purkinje images in accordance with certain embodiments.

FIG. 12 illustrates an example computing device that implements, at least partly, one or more functionalities for facilitating visualization during ophthalmic surgery in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

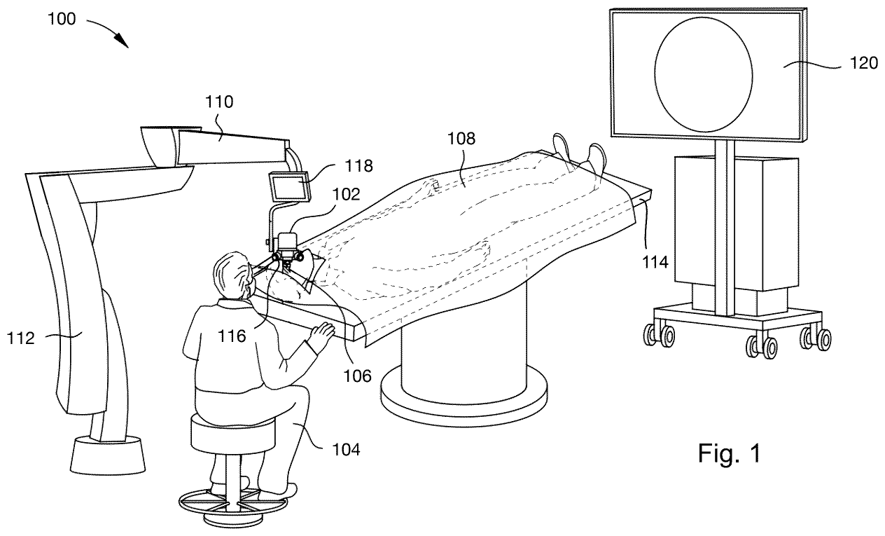

FIG. 1 illustrates an example ophthalmic system 100 with which ophthalmic treatments are performed in an operating environment. The system 100 includes an ophthalmic microscope 102. A surgeon 104 uses the ophthalmic microscope 102 to visualize structures on and in an eye 106 of a medical patient 108 undergoing surgery. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices, which are viewable through binocular eyepieces 116 and display images of the patient's eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may additionally or alternatively be displayed on one or more display devices in the operating environment. For example, the one or more display devices may include a display device 118 fastened to the supporting arm 110 above the ophthalmic microscope 102.

In order to relieve the surgeon 104 from the need to constantly look into the eye pieces 116 to obtain a stereoscopic view, the one or more display devices may also include a display device 120 that can be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

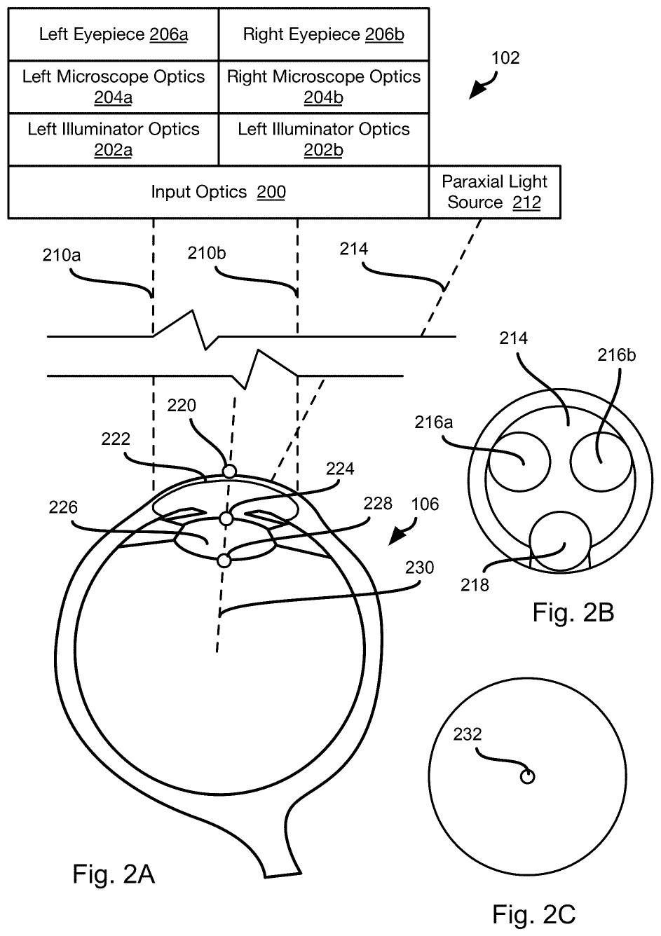

FIG. 2A is schematic diagram of the ophthalmic microscope 102, which includes input optics 200, left and right illuminator optics 202a, 202b, left and right microscope optics 204a, 204b, and left and right eye pieces 206a, 206b. As used herein, “left” and “right” are used to refer to first and second instances of components facilitating visualization by the left and right eyes of the surgeon 104. The use of “left” and “right” shall be understood as exemplary only and it shall be understood that these can be readily interchanged without change in functionality.

The input optics 200 receive light reflected from the eye 106 of the patient. The input optics 200 may include a set of lenses with a common optical axis or two sets of lenses with offset and/or non-parallel optical axes, e.g., right and left sets of lenses. The left and right illuminator optics 202a, 202b include light sources and optics that both (a) direct light from the light sources onto the eye 106 and (b) permit light reflected from the eye to pass through the illuminator optics 202a, 202b. The left and right illuminator optics 202a, 202b may therefore each include a beam splitter and possibly one or more lenses to facilitate this function. The light sources of the left and right illuminator optics 202a, 202b may be embodied as light emitting diodes (LED) or other light sources. The light sources may be operable at a variety of intensities and colors. In certain embodiments, the light sources may include LEDs having three different wavelength distributions (e.g., centered on red, green, and blue wavelengths) and having independently selectable intensities such that the color emitted by the light source may be controlled. The light sources may additionally or alternatively include infrared or near-infrared light sources enabling one or both (a) illumination using light that is not visible to the patient and (b) illumination using visible light that causes very little patient discomfort.

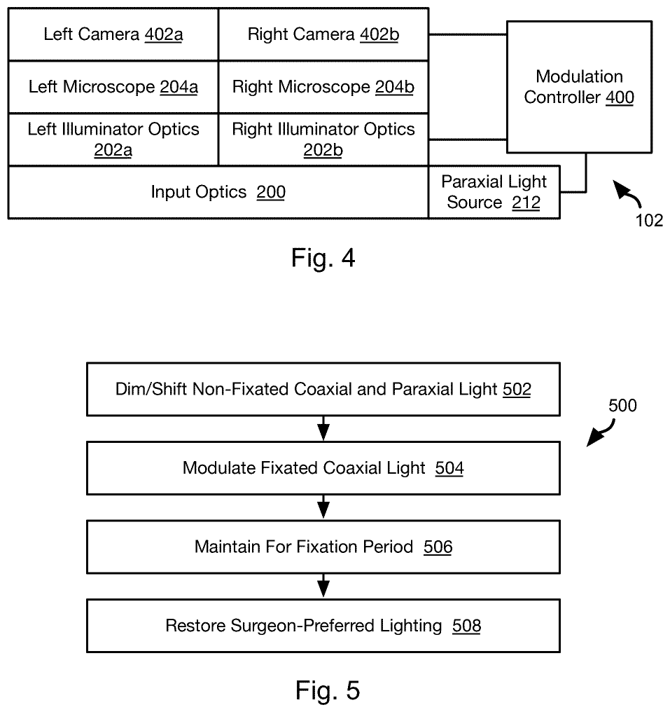

Light reflected from the eye 106 passes through the left and right illuminator optics 202a, 202b and is magnified by left and right microscope optics 204a, 204b, respectively. The magnification of the left and right microscope optics 204a, 204b may be adjustable. Likewise, the depth of focus of the left and right microscope optics 204a, 204b may be adjustable. Light output reflected from the eye is emitted by the left and right microscope optics 204a, 204b through left and right eye pieces 206a, 206b, respectively, for viewing by the surgeon 104. The left and right eye pieces 206a, 206b may include soft (e.g., rubber) interfaces for contacting the surgeon's 104 face and possibly one or more output lenses. The eye pieces 206a, 206b may be replaced with or include cameras for capturing images output by the left and right microscope optics 204a, 204b (see FIG. 4 and associated description).

Light from the illuminator optics 202a, 202b is emitted onto the eye 106. The light is directed along the optical axes 210a, 210b of the left and right sides of the ophthalmic microscope 102, i.e., the optical axes 210a, 210b of the left and right illuminator optics 202a, 202b. The optical axes 210a, 210b may be parallel to one another or converge at a point outward from the input optics 200. In some embodiments, additional illumination is provided by a paraxial light source 212 directed along axis 214 that is non-parallel with respect to the optical axes 210a, 210b, such as at an angle of between 5 and 12 degrees. As used herein, the light from the left and right illuminator optics 202a, 202b is referred to as “left coaxial light,” “right coaxial light,” or collectively as “coaxial lights.” The light from the paraxial light source 212 is referred to as “paraxial light.”

FIG. 2B illustrates an example appearance of the ophthalmic microscope 102 to the eye 106 of the patient. The light emitted by the left and right illuminator optics 202a, 202b appears as two bright spots 216a, 216b and the light from the paraxial light source 212 appears as a third bright spot 218 offset from the bright spots 216a, 216b.

Turning back to FIG. 2A, the light from the left and right illuminator optics 202a, 202b and the paraxial light source 212 are incident on the eye 106 and portions thereof reflect off of different surfaces of the eye 106. For example, a portion 220 of the light reflects from the anterior surface of the cornea 222 and is referred to as the P1 Purkinje image. A portion also reflects from the posterior surface of the cornea and is referred to as the P2 Purkinje image but is less discernable and is typically not used. A portion 224 of the light reflects from the anterior surface of the crystalline lens 226 and is referred to as the P3 Purkinje image. A portion 228 of the light reflects from the posterior surface of the crystalline lens 226 and is referred to as the P4 Purkinje image. In practice, the P1 and P4 Purkinje images are the most visible and used clinically.

Referring to FIG. 2C, the visual axis 230 of the eye 106 may be oriented such that the P1 and P4 Purkinje image for a specific light source overlap completely, resulting in a single visible image 232. When the P1 and P4 Purkinje images of a light source (left illuminator optics 202a, right illuminator optics 202b, or the paraxial light source 212) are aligned, the visual axis 230 of the eye 106 is aligned with the optical axis of that light source. In practice, since there are three light sources in the illustrated configuration, there will be three sets of P1 and P4 Purkinje images, one of which can be aligned at a time.

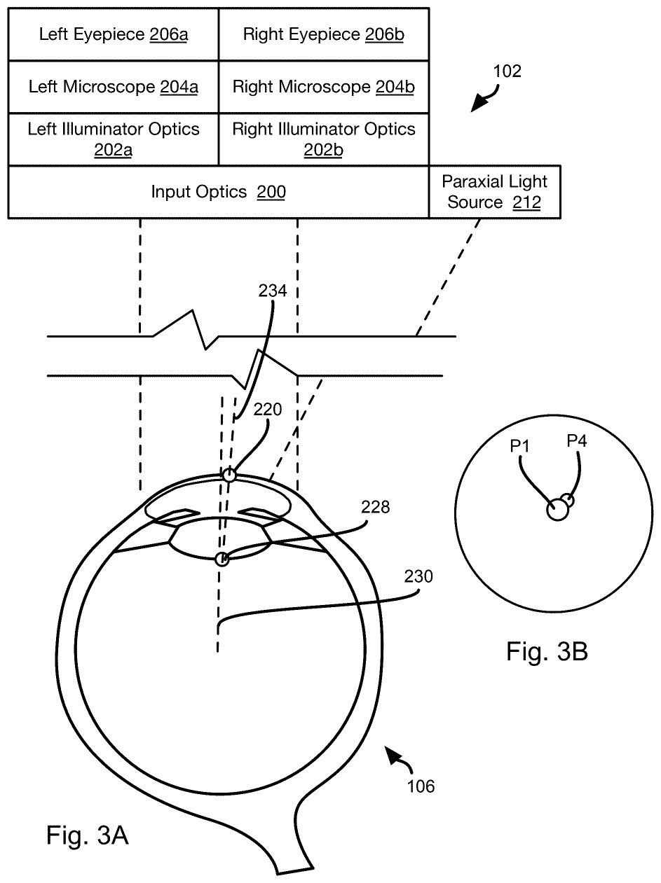

Referring to FIGS. 3A and 3B, when the visual axis 230 of the eye 106 is not aligned with an axis 234 intersecting the P1 and P4 Purkinje images (FIG. 3A), the P1 and P4 images for a single light source will appear offset from one another (FIG. 3B).

In prior approaches, prior to performing an ophthalmic treatment, the surgeon 104 would direct the patient to fixate on one of the spots 216a, 216b in order to align the eye 106 with the ophthalmic microscope 102 and identify the visual axis 230 of the eye 106. The surgeon would verify alignment by noting the degree of overlap between the P1 and P4 Purkinje images. The surgeon 104 would then perform an ophthalmic treatment, such as phacoemulsification, followed by implantation of an intraocular lens (IOL). The surgeon would also instruct the patient to fixate on one of the spots 216a, 216b following implantation of an IOL to assess tilt of the lens based on two or more of the P1, P3, and P4 Purkinje images.

However, relying on the patient to voluntarily fixate is difficult because the multiple light sources can cause confusion as to which light to fixate on. Likewise, a patient may have difficulty fixating on one of the spots 216a, 216b for some other reason. In addition, the intensity of the spots 216a, 216b, 218 required for visualization by the surgeon may cause discomfort to the patient during fixation.

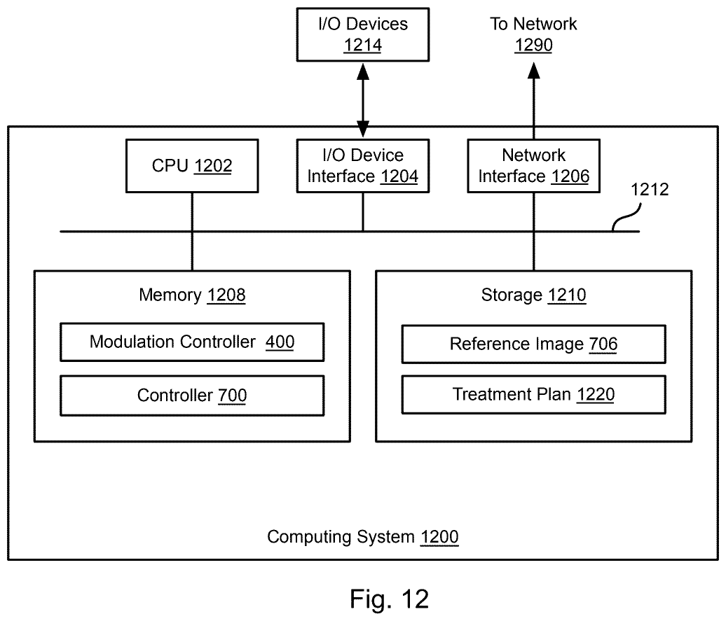

FIGS. 4 and 5 illustrate an improved approach for aligning the visual axis 230 with respect to a light source using Purkinje images of that light source. Referring specifically to FIG. 4, the right and left illuminator optics 202a, 202b may be coupled to a modulation controller 400. The modulation controller 400 may further receive images output by a left camera 402a and a right camera 402b. Alternatively, in some embodiments, the ophthalmic microscope 102 may lack cameras and use left and right eye pieces 206a, 206b as with the embodiment of FIG. 2A. The left and right cameras 402a, 402b may detect light output by the left and right microscope optics 204a, 204b, respectively. The modulation controller 400 may be implemented by a computing system, such as the computing system 1200 of FIG. 12. The modulation controller 400 may control the right and left illuminator optics 202a, 202b and paraxial light source 212 in order to enhance fixation on and visualization of Purkinje images.

FIG. 5 illustrates a method 500, which, for example, may be performed by the modulation controller 400 of FIG. 4. In some embodiments, the method 500 does not include the use of images. The method 500 presumes that one of the left and right illuminator optics 202a, 202b is a “fixated light,” i.e., selected by default or by the surgeon as the light upon which the patient is to fixate. For example, the left illuminator optics 202a may be assumed to be the fixated light, while the right illuminator optics 202b and the paraxial light source 212 are the non-fixated lights.

The method 500 may include performing, at step 502, at least one of dimming or shifting the wavelength of the non-fixated lights. Shifting the wavelength may include controlling current supplied to LEDs of different colors (of the non-fixated lights) to obtain a different wavelength distribution. The degree of dimming may include reducing intensity of the non-fixated lights by at least 25 percent or at least 50 percent of the intensity of each non-fixated light relative to “surgeon-preferred lighting,” i.e., lighting intensity and wavelength distributions of the left and right illuminator optics 202a, 202b and the paraxial light source 212 selected by the surgeon to provide a desired degree of visibility. Dimming may be performed to enhance patient comfort and reduce phototoxicity. Likewise, the shifting may include shifting the wavelength distribution toward a longer range of wavelengths, e.g., shifting the center (highest intensity) wavelength of the distribution up by at least 20, 50, or 100 nanometers.

The method 500 may further include performing, at step 504, modulation of the fixated light to enhance visibility and patient comfort. Modulating of the fixated light may include modulating the wavelength of the fixated light to distinguish the fixated light from the non-fixated lights. For example, the wavelength distribution may be varied abruptly or sinusoidally (e.g., digital approximation of sinusoidally) between two different wavelength distributions. Modulating of the fixated light may include pulsing the intensity of the fixated light, e.g., abruptly or sinusoidally varying the intensity of the fixated light. The modulation of the fixated light at step 504 enhances fixation by some or all of (a) clearly indicating which of the lights to fixate upon, (b) reducing the amount of light entering the patient's eye over time, and (c) enabling the surgeon 104 to readily identify which Purkinje image to observe to assess alignment. Modulation at step 504 of wavelength and/or intensity may be at a frequency that enhances visibility to the eye 106 of the patient, such as a frequency of between 2 and 20 Hz.

The settings for the non-fixated lights and the fixated light from steps 502 and 504 may be maintained, at step 506, for a fixation period. The fixation period may be a predetermined amount of time, or may end upon receiving an input from the surgeon 104. Likewise, the start of the fixation period may be invoked in response to an input from the surgeon 104. Inputs from the surgeon may be received through a button, touch screen, microphone (e.g., detecting voice commands), or camera (e.g., detecting gestures) coupled to the ophthalmic microscope 102. Invoking and/or ending of the fixation period may also be specified in, and controlled based on, a treatment plan uploaded to the computing device implementing the modulation controller 400. Following elapse of the fixation period, the method 500 may include restoring, at step 508, the surgeon-preferred lighting. The method 500 may be repeated either (a) on a fixed period, (b) when instructed by an input from the surgeon, or (c) when directed by the treatment plan.

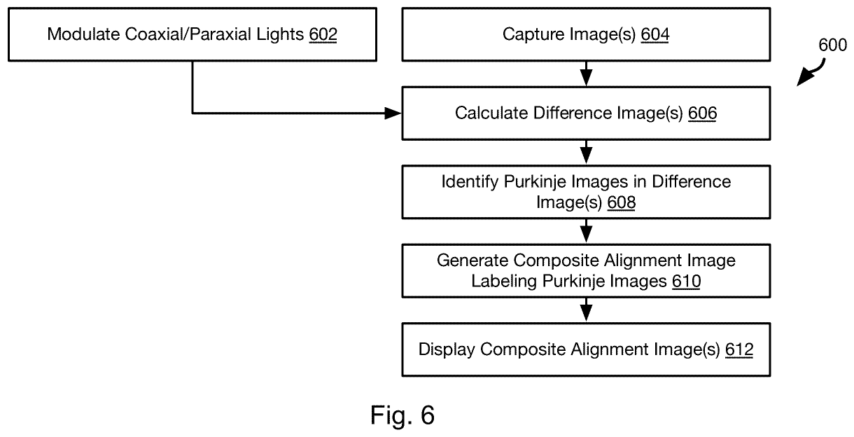

FIG. 6 illustrates a method 600 that may be performed by the modulation controller 400 and that includes the use of images from one or both of the left and right cameras 402a, 402b. The method 600 includes modulating, at step 602, some or all of the left coaxial light, right coaxial light, and the paraxial light. Modulation of intensity or wavelength may include sinusoidal or abrupt modulation. Modulation, which is described in more detail below, may be performed in synchronization with a frame rate of one or both of the left and right cameras 402a, 402b. In particular, for each light of the left coaxial, right coaxial, and paraxial light, there may be a transition between states (e.g., between first and second states) before capture of a next image frame by one or both of the left and right cameras 402a, 402b. Each state of the states of each light may have a different intensity and/or wavelength distribution than other states. The change in states may be abrupt, such as changing in intensity at at least 80, 90, or 95 percent of the rate at which the light source is capable of changing intensity. The change in states may also be sinusoidal with a period equal to the frame rate.

In one example, the modulation performed at step 602 may include implementing a pattern for each of the fixated and non-fixated lights. For example, the pattern may include N time steps, e.g., N consecutive periods of the frame rate. For example, for the fixated light, the pattern may include causing the fixated light to transition to a second state every Nth frame with all other frames being at a first state, where N is an integer greater than 1, such as from 2 to 100. For example, N may be selected such that one frame every X seconds is illuminated at the second state, where X is N times the frame period of the left and right cameras 402a, 402b. For example, X may be from 0.5 to 2 seconds, such as 1 second. The modulation pattern may be selected to be imperceptible or at least unlikely to cause fatigue from flickering. For example, the fixated light may drop in intensity or be turned off completely every Nth frame. A more complex pattern may be used: high, low, low, high, low, high, etc., where the fixated light has higher intensity during “high” time steps than in “low” time steps. The non-fixated lights may either (a) both be modulated with a pattern that is different from that used for the fixated light, (b) modulated with different patterns from one another and the fixated light, or (c) not be modulated at all, e.g., constant intensity and/or wavelength within the capabilities of the light source. In some embodiments, at least 50 percent, at least 75 percent, or at least 80 percent of the images are captured with the surgeon preferred lighting.

The method 600 may further include capturing, at step 604, images of the eye 106 using one or both of the left and right cameras 402a, 402b. Step 604 may include capturing an image at each time step of the pattern implemented at step 602 using one or both of the left and right cameras 402a, 402b.

The method 600 may include calculating, at step 606, difference image(s) based on the images captured at step 606. For example, left and right difference images may be calculated for images captured using the left and right cameras 402a, 402b, respectively. Each difference image may be a function of the pattern. For example, suppose the pattern includes N1 first images captured with lighting in the first state and N2 second images captured with lighting in the second state, where N1 and N2 are integers such that N1+N2=N. The difference image may therefore include computing a pixelwise summation of the pixels of the first images to obtain a first aggregate image (A1), computing a pixelwise summation of the pixels of the second images to obtain a second aggregate image (A2), and calculating the difference image D by computing a pixelwise difference of A1 and A2: D[n,m]=A2[n,m]/N2−A1[n,m]/N1), wherein n and m are the indexes of pixel positions within the first and second aggregate images A1, A2. As used herein, a “pixelwise” operation (addition, subtraction, division, multiplication, etc.) with respect to one or more images shall be understood as including performing the pixelwise operation at each pixel position with respect to pixel values of the one or more images at the each pixel position. In the case where N1 or N2 is equal to 1, then no aggregation is performed and A1 or A2 is simply a first frame captured in the first state or a second frame captured in the second state, respectively.

The difference image(s) from step 606 includes non-zero values only for changes between A1 and A2. Due to the correspondence between the modulation of lighting according to step 602 and the calculation of the difference image, changes in pixels due to changes in lighting will be highlighted. Accordingly, the Purkinje images generated by reflections of the fixation light will be readily visible. In some instances, the Purkinje images in the difference image(s) may be the only non-zero pixels, or pixels above some minimum intensity threshold, in the different image(s). Accordingly, pixels below the minimum intensity threshold may be clamped to zero.

The method 600 may further include identifying, at step 608, the Purkinje images in the difference image(s). For example, the Purkinje images may be identified in each difference image. Corresponding pixels in one or more of the alignment images may be labeled to generate, at step 610, a composite alignment image. For example, corresponding pixels in the composite image may be made brighter, changed to an artificial color that contrasts with the tissue of the eye, marked with a symbol, or otherwise highlighted. Misalignment may be represented in other ways, such as an arrow, text, or other indicator that is added to the composite image and which indicates a direction that the eye 106 must move relative the ophthalmic microscope 102 or that the ophthalmic microscope must move relative to the eye 106.

The composite image may then be displayed at step 612, such as on one or both of the display devices 118, 120, on a display device internal to the ophthalmic microscope 102, or other display device. The method 600 may be repeated throughout a surgery constantly, upon receiving an input from the surgeon, or as specified in a treatment plan.

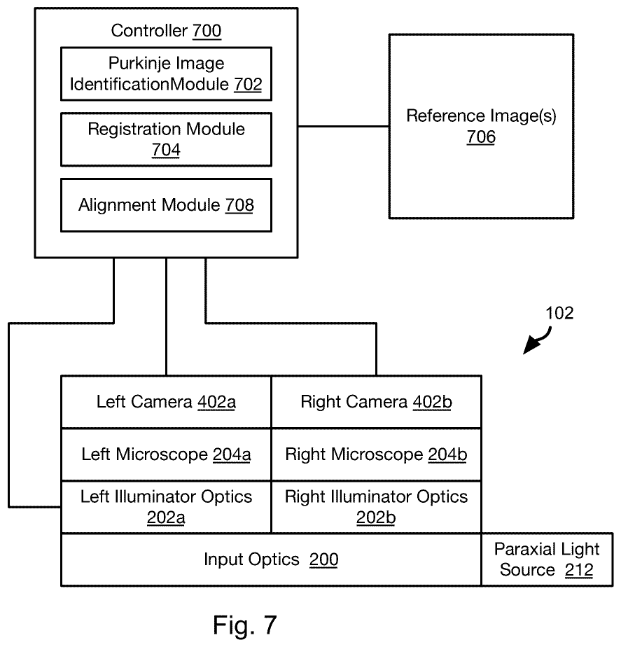

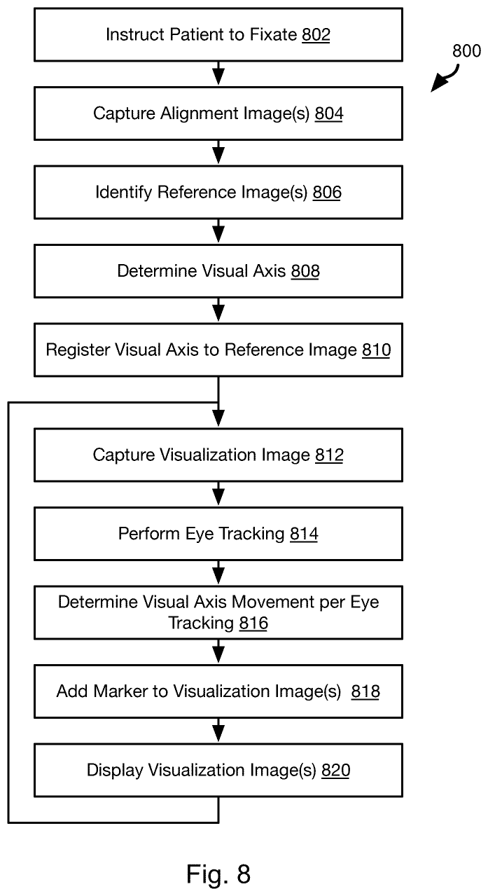

Referring to FIGS. 7 and 8, in an alternative approach, following initial alignment using Purkinje images, subsequent alignment, or assessment of misalignment, is performed without using Purkinje images. Referring specifically to FIG. 7, a controller 700 may be coupled to the left and right cameras 402a, 402b and receive images output thereby. The controller 700 may store and access one or more reference images 706 as described in greater detail below. The controller 700 maybe implemented by a computing system 1200 as described below. The controller 700 may additionally be coupled to the left and right illuminator optics 202a, 202b

The controller 700 may include a Purkinje image identification module 702 that is configured to identify the Purkinje images, such as the P1 and P4 Purkinje images. The Purkinje image identification module 702 may implement the approach described above with respect to FIG. 6 using a machine learning model (e.g., convolution neural network (CNN)) trained to identify the Purkinje images

The controller 700 may include a registration module 704. The registration module 704 associates the visual axis 230 of the eye (as indicated by the Purkinje images) with one or more reference images 706 received from the left and right cameras 402a, 402b. For example, a first image from one camera, e.g., the left camera 402a, having the Purkinje images aligned may be stored as a first reference image. In some examples, the first image may include a label of the Purkinje images. Likewise, a second image from the other camera, e.g., the right camera 402b, may be stored as a second reference image. In some examples, the second image may include a label of the Purkinje images. In this and other examples disclosed herein, the roles of the right and left cameras 402a, 402b may be reversed.

Accordingly, when the image from the left camera 402a matches the first reference image and the image from the right camera 402b matches the second reference image, it can be inferred that the eye's visual axis 230 is aligned as the visual axis 230 was aligned when the first and second reference images were captured. Alignment may therefore be assessed without subsequent identification of the Purkinje images.

The controller 700 may further include an alignment module 708. Due to the binocular vision inherent in the system, when the first and second reference images match the images from the left and right cameras 402a, 402b, it can be inferred that the visual axis 230 of the eye is aligned in three-dimensional space. Accordingly, the alignment module 708 may assess alignment of current images from the left and right cameras 402a, 402b in order to determine misalignment of the visual axis 230 of the eye 106. For example, the alignment module 708 may use an eye tracking algorithm to determine deviation of the position and/or orientation of the eye 106 relative to the first and second reference images. Note that in some embodiments, a single reference image may be used and eye tracking may be performed in a like manner to determine deviation of the eye 106 from the position and/or orientation of the eye at the time of capture of the reference image.

FIG. 8 illustrates a method 800 that may be executed, at least in part, by the controller 700. The method 800 may include instructing, at step 802, a patient to fixate on the fixation light. Step 802 may be accompanied by performance of the method 500 to facilitate fixation.

At step 804, alignment images are captured using one or both of the left and right cameras 402a, 402b. “Alignment images” may be understood as images captured for purposes of generating the one or more reference images 706 described above and may be captured with lighting other than the surgeon-preferred lighting. The alignment images may be a series of consecutive video images.

At step 806, the one or more reference images are identified from the alignment images. The one or more reference images may be identified as having two or more Purkinje images aligned with one another, such as by using the approach described with respect to FIG. 6 or using a machine learning model trained to perform this task. The Purkinje images determined to be aligned may include the P1 and P4 Purkinje images. For example, step 806 may include identifying an image of the alignment images in which the two Purkinje images (e.g., P1 and P4) are aligned, e.g., appear as a single spot, as a first reference image. For example, where the fixation light is the left illuminator optics 202a, step 806 may include identifying the image in which the two Purkinje images are aligned from among the alignment images received from the left camera 402a. Step 806 may include identifying a corresponding image from the other camera, e.g., the right camera 402b in this example, as the second reference image, the corresponding image being captured simultaneously (e.g., within less than 10 percent of the frame rate or within less than 10 milliseconds) with the first reference image.

The method 800 may include determining, at step 808, the visual axis 230 of the eye 106. Step 808 may include identifying the same two Purkinje images in the second reference image. Using stereoscopic vision techniques and the positions of the two Purkinje images in the first and second reference images, the three-dimensional position and orientation of the visual axis may be determined. For example, step 808 may include identifying the three-dimensional positions of the two Purkinje images in the first and second reference images and defining the visual axis as passing through the two three-dimensional positions.

The method 800 may include registering, at step 810, the visual axis with respect to the first and second reference images. For example, three or more visible features of the eye may be identified in the first and second reference images, and the three-dimensional positions of these features may further be identified. The visual axis may then be defined with respect to the three-dimensional positions of these features. Alternatively, a three-dimensional volumetric model of the eye may be generated from the first and second reference images with the visual axis being defined as a line within the coordinate system of the three-dimensional volumetric model.

The method 800 may thereafter include the use of the first and second reference images and/or the registration of the visual axis with respect to the first and second reference images.

For example, the method 800 may include capturing, at step 812, visualization images from the left and right cameras 402a, 402b. As used herein “visualization images” may be images captured using the surgeon-preferred lighting.

The method 800 may include performing, at step 814, eye tracking with respect to the images captured at step 812. Step 814 may be performed using any eye tracking algorithm known in the art. Step 814 may include detecting three-dimensional change(s) in position and/or orientation of the eye 106 with respect to the surgical microscope 102.

The method 800 may further include determining, at step 816, movement of the visual axis of the eye 106 according to the eye tracking. For example, step 816 may include determining movement of the eye 106 relative to the position of the eye 106 represented in the reference images. For example, for a given change in position of the eye 106 indicated by the eye tracking, the corresponding change in the visual axis may be determined, such as by applying the same transformation (change in angle and/or translation) to the visual axis.

The method 800 may include adding, at step 818, a marker to the visualization images. The marker may be a representation of the current position of the visual axis, e.g., a two-dimensional rendering of a three-dimensional line representing the visual axis from the point of view of the left and right cameras 402a, 402b, resulting in perception of the three-dimensional line by the surgeon 104. Step 818 may include providing one or more arrows, text indicators, or other symbol, indicating a direction and/or magnitude to rotate the eye 106 to achieve alignment of the visual axis with the position determined at step 808. Step 818 may include outputting text describing movement required to achieve alignment of the visual axis with the position determined at step 808.

The visualization images having the marker from step 818 added thereto may then be displayed at step 820. For example, the visualization images may be displayed on one or both of the display devices 118, 120, on a display device internal to the ophthalmic microscope 102, or other display device. Steps 812-820 may be repeated throughout a surgery constantly, upon receiving an input from the surgeon, or as specified in a treatment plan.

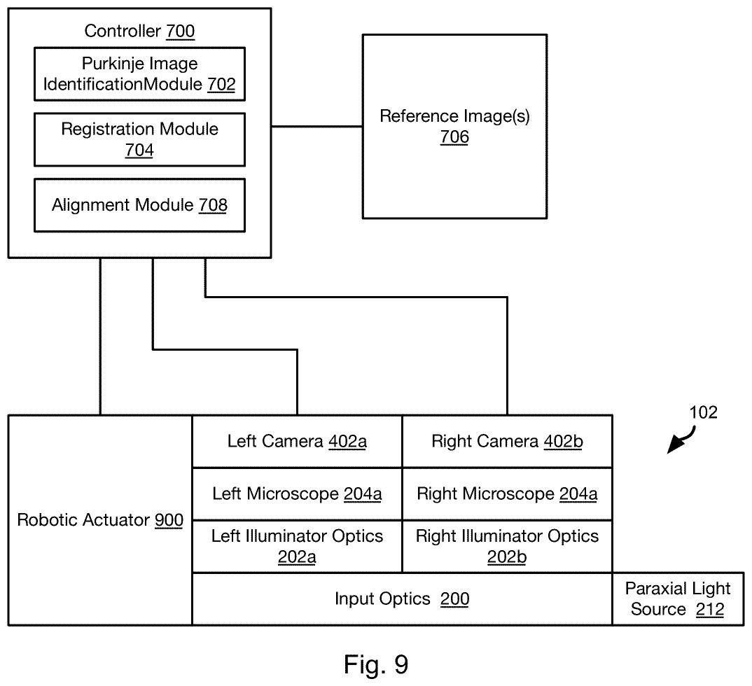

Referring to FIG. 9, in some embodiments, the ophthalmic microscope 102 is coupled to a robotic actuator 900. The robotic actuator may have at least four degrees of freedom (DOF), such as at least two translational degrees of freedom (DOF) and at least two rotational DOF. In some other embodiments, the robotic actuator has five or six DOF. The robotic actuator may be embodied as a serial robotic arm, gantry, or other type of robotic actuator.

The controller 700 may be further coupled to the robotic actuator 900. For example, the controller 700 may cause the robotic actuator to move the ophthalmic microscope 102 in correspondence with changes in the position and/or orientation of the visual axis 230 in order to maintain alignment of the visual axis 230 with respect to the ophthalmic microscope 102.

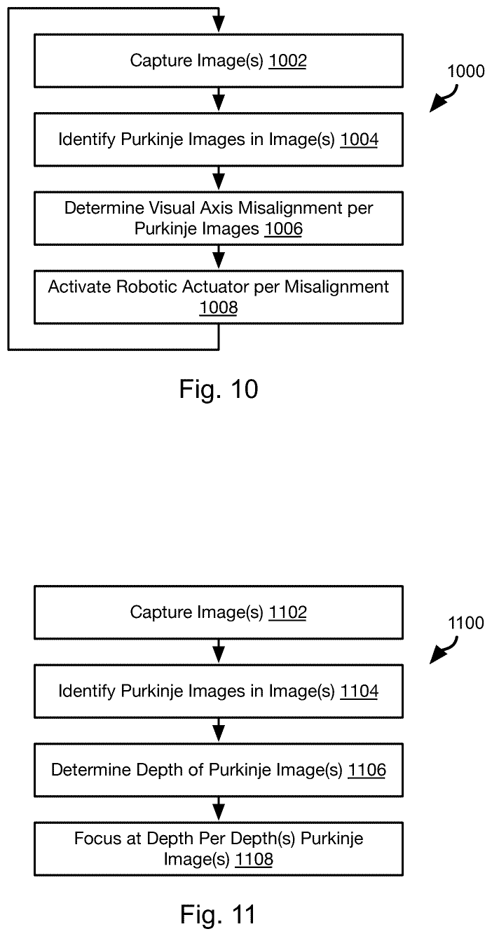

Referring to FIG. 10, the controller 700 may use the robotic actuator 900 to implement the illustrated method 1000. The method 1000 may include capturing, at step 1002, images using the right and left cameras 402a, 402b. The Purkinje images, such as P1 and P4, are identified at step 1004. Step 1004 may be implemented using the approach of FIG. 6, or using a machine learning model trained to perform this task. The method 1000 includes determining, at step 1006, misalignment of the optical axis based on the Purkinje images. For example, the misalignment of the Purkinje images in the images from the right and left cameras 402a, 402b may be processed to obtain the three-dimensional positions of the Purkinje images. Likewise, the misalignment of the Purkinje images in the image corresponding to the fixation light (e.g., the left camera 402a where the left illuminator optics 202a provide the fixation light) indicates the direction and amount of misalignment.

The method 1000 may include activating, at step 1008, the robotic actuator 900 to drive the misalignment toward zero. In a first case, the robotic actuator 900 moves only in two dimensions in correspondence with misalignment with the P1 and P4 Purkinje images, i.e., a direction of movement corresponding to a vector pointing from P4 to P1. Such movement may continue until P4 and P1 are aligned.

In a second case, a three-dimensional representation of the visual axis 230 is obtained from the representations of P1 and P4 in the images from the right and left cameras 402a, 402b. The robotic actuator 900 is actuated to align the optical axis of the fixation light, e.g., the optical axis of the left illuminator optics 202a, to align with the visual axis 230.

In yet another example, the misalignment of the visual axis 230 may be determined as described above with respect to FIG. 8. Accordingly, step 1008 may include activating the robotic actuator 900 to drive the optical axis of the fixation light into alignment with the visual axis 230 as determined according to FIG. 8.

Referring to FIG. 11, any of the embodiments of the ophthalmic microscope 102 described herein may additionally use the Purkinje images detected in images from the cameras 402a, 402b to automatically set the focal plane of the ophthalmic microscope 102 according to method 1100. In particular, common focal planes are the cornea or the iris of the eye 106. The Purkinje points are tied to depths along the visual axis 230 and therefore may be used as reference points to automatically focus the ophthalmic microscope 102.

For example, method 1100 may include capturing, at step 1102, images using the cameras 402a, 402b and identifying, at step 1104, one or more Purkinje images in the captured images. The Purkinje images may be identified using the approach described above with respect to FIG. 6 or a machine learning model trained to perform this task. The depth of each Purkinje image may be determined at step 1106. In particular, using stereoscopic imaging techniques, the depth of a Purkinje image may be determined from the location of the Purkinje image in images from the left and right cameras 402a, 402b.

The method 1100 may then include adjusting, at step 1108, the left and right microscope optics 204a, 204b such that the ophthalmic microscope 102 is focused at a depth selected according to the depths of the Purkinje images determined at step 1106. For example, where the cornea is the desired focal plane, the depth of focus may be set to the depth of the P1 Purkinje image. Where the iris is the desired focal plane, the depth of focus may be set at an intermediate depth between the depths of the P1 and P4 Purkinje images, such as using a typical (e.g., experimentally measured average) location of the iris relative to the anterior surface of the cornea 222 and the posterior surface of the lens 226. The measured locations of anatomy may be measured using optical coherence tomography (OCT), scanning laser ophthalmoscope, or other measurement technique. In some embodiments, the relative depths of the P1 and P4 Purkinje images may be used to approximate the anterior chamber depth (ACD) of the eye 106 (e.g., a difference between the depths of P1 and P4 or a result of some other computation).

FIG. 12 illustrates an example computing system 1200. The ophthalmic microscope 102 and/or the display device 120 may incorporate a computing device having some or all of the attributes of the computing system 1200.

As shown, computing system 1200 includes a central processing unit (CPU) 1202, one or more I/O device interfaces 1204, which may allow for the connection of various I/O devices 1214 (e.g., keyboards, displays, mouse devices, pen input, etc.) to computing system 1200, network interface 1206 through which computing system 1200 is connected to network 1290, a memory 1208, storage 1210, and an interconnect 1212.

CPU 1202 may retrieve and execute programming instructions stored in the memory 1208. Similarly, CPU 1202 may retrieve and store application data residing in the memory 1208. The interconnect 1212 transmits programming instructions and application data, among CPU 1202, I/O device interface 1204, network interface 1206, memory 1208, and storage 1210. CPU 1202 is included to be representative of a single CPU, multiple CPUs, a single CPU having multiple processing cores, and the like.

Memory 1208 is representative of a volatile memory, such as a random access memory, and/or a nonvolatile memory, such as nonvolatile random access memory, phase change random access memory, or the like. As shown, memory 1208 may store executable code implementing one or both of the modulation controller 400 and the controller 700.

Storage 1210 may be non-volatile memory, such as a disk drive, solid state drive, or a collection of storage devices distributed across multiple storage systems. Storage 1210 may optionally store a reference image 706 or a treatment plan 1220, such as a treatment plan as described above.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic system comprising:
an ophthalmic microscope including first illuminator optics and second illuminator optics configured to emit light onto an eye of a patient; and
a controller coupled to the first illuminator optics and the second illuminator optics, the controller configured:
operate in a first mode in which light emitted by the first illuminator optics and light emitted by the second illuminator optics have a first configuration; and
operate in a second mode in which the light emitted by the first illuminator optics and the light emitted by the second illuminator optics have a second configuration that (a) enhances visibility of one or more Purkinje images projected onto the eye of the patient by the first illuminator optics relative to the first configuration.

2. The system of claim 1, wherein the controller is further configured to operate in the second mode such that the second configuration (b) enhances fixation by the patient on the light emitted by the first illuminator optics relative to the first configuration.

3. The system of claim 2, wherein the controller is configured to achieve (a) and (b) by changing a wavelength distribution of the light emitted by the first illuminator optics relative to the first configuration.

4. The system of claim 3, wherein the controller is configured to achieve (a) and (b) by shifting the wavelength distribution toward longer wavelengths.

5. The system of claim 2, wherein the controller is configured to achieve (a) and (b) by changing an intensity of the light emitted by the first illuminator optics relative to the first configuration.

6. The system of claim 2, wherein the controller is configured to achieve (a) and (b) by modulating an intensity of the light emitted by the first illuminator optics at a frequency of between 2 and 20 Hz.

7. The system of claim 2, wherein the controller is configured to achieve (a) and (b) by changing a wavelength of the light emitted by the second illuminator optics relative to the first configuration.

8. The system of claim 2, wherein the controller is configured to achieve (a) and (b) by decreasing intensity of the light emitted by the second illuminator optics relative to the first configuration.

9. The system of claim 2, further comprising a paraxial light source offset from optical axes of the first illuminator optics and the second illuminator optics and defining an angle relative to the optical axes of between 5 and 12 degrees, wherein the controller is further configured to achieve (a) and (b) by changing at least one of intensity and wavelength distribution of light emitted by the paraxial light source relative to the first configuration.

10. The system of claim 1, wherein the one or more Purkinje images include P1 and P4 Purkinje images.

11. An ophthalmic system comprising:
an ophthalmic microscope including:
first illuminator optics, first microscope optics, and a first camera, the first illuminator optics configured to emit first light onto an eye of a patient and transmit first reflected light from the eye of the patient to the first microscope optics, the first camera configured to detect at least a portion of the first reflected light that passes through the first microscope optics; and
second illuminator optics, second microscope optics, and a second camera, the second illuminator optics configured to emit second light onto the eye of the patient and transmit second reflected light from the eye of the patient to the second microscope optics, the second camera configured to detect at least a portion of the second reflected light that passes through the second microscope optics; and
a controller coupled to the first illuminator optics, the second illuminator optics, the first camera, and the second camera, the controller configured to:
(a) modulate at least one of the first light or the second light to enhance visibility of Purkinje images projected onto the eye of the patient by the first illuminator optics;
while performing (a), capture visualization images output by the first camera and the second camera;
identify locations of the Purkinje images in the visualization images; and
produce an output based on the locations.

12. The system of claim 11, wherein the controller is configured to perform (a) by modulating intensity of the first light according to a pattern synchronized with a frame rate at which the visualization images are captured.

13. The system of claim 12, wherein the controller is configured to calculate one or more difference images from the visualization images in accordance with the pattern and identify locations of the Purkinje images in the one or more difference images.

14. The system of claim 13, wherein the pattern includes one or more first images captured with the first light at a first intensity and one or more second images captured with the second light at a second intensity that is less than the first intensity, each difference images calculated as a pixelwise difference between at least one of the one or more first images and at least one of the one or more second images.

15. The system of claim 11, wherein the output comprises a visualization labeled with the locations of the Purkinje images.

16. The system of claim 11, further comprising a robotic actuator coupled to the ophthalmic microscope,
wherein the output comprises activation of the robotic actuator.

17. The system of claim 11, wherein the output comprises an adjustment of a foci of the first microscope optics and the second microscope optics according to the locations.

18. A system comprising:
an ophthalmic microscope including:
first illuminator optics, first microscope optics, and a first camera, the first illuminator optics configured to emit first light onto an eye of a patient and transmit first reflected light from the eye of the patient to the first microscope optics, the first camera configured to detect at least a portion of the first reflected light that passes through the first microscope optics; and
second illuminator optics, second microscope optics, and a second camera, the second illuminator optics configured to emit second light onto the eye of the patient and transmit second reflected light from the eye of the patient to the second microscope optics, the second camera configured to detect at least a portion of the second reflected light that passes through the second microscope optics; and
a controller coupled to the first illuminator optics, the second illuminator optics, the first camera, and the second camera, the controller configured to:
receive first images of the eye of the patient from the first camera and the second camera;
identify Purkinje images in first images output by the first camera and the second camera;
registering a visual axis defined by the Purkinje images with respect to the first images;
receive second images of the eye of the patient from the first camera and the second camera after receiving the first images;
perform eye tracking to determine movement of the eye of the patient relative to a position of the eye at a time of capture of the first images;
determine a current position of the visual axis according to the eye tracking; and
produce an output based on the current position.

19. The system of claim 18, wherein the output comprises a marker indicating misalignment of the eye of the patient.

20. The system of claim 18, further comprising a robotic actuator coupled to the ophthalmic microscope,
wherein the output is an activation of the robotic actuator selected to drive the ophthalmic microscope to toward a position relative to the eye of the patient corresponding to the first images.
